(19) United States
(12) Patent Application Publication
(10) Pub. No.: US 2014/0051163 A1
(43) Pub. Date: Feb. 20, 2014

(54) Synthetic Substrate for Stem Cell Culture and Methods of Use Thereof

(71) Applicants: Kevin E. Healy, Moraga, CA (US); Elizabeth F. Irwin, San Francisco, CA (US)

(72) Inventors: Kevin E. Healy, Moraga, CA (US); Elizabeth F. Irwin, San Francisco, CA (US)

(73) Assignee: THE REGENTS OF THE UNIVERSITY OF CALIFORNIA, Oakland, CA (US)

(21) Appl. No.: 13/982,848

(22) PCT Filed: Feb. 1, 2012
(86) PCT No.: PCT/US12/23527

§ 371 Date: Oct. 28, 2013

Related U.S. Application Data
(60) Provisional application No. 61/439,677, filed on Feb. 4, 2011.

(51) Int. Cl. C12N 5/0735

(52) U.S. Cl. CPC C12N 5/0606
USPC 435/366; 435/396; 435/363; 525/296

(57) ABSTRACT

The present disclosure provides synthetic substrates for long-term culture of stem cells; and methods of use of the synthetic substrates.

### CROSS-REFERENCE

This application claims the benefit of U.S. Provisional Patent Application No. 61/439,677, filed Feb. 4, 2011, which application is incorporated herein by reference in its entirety.

### STATEMENT REGARDING FEDERALLY SPONSORED RESEARCH

This invention was made with government support under Grant No. HL096525-01 awarded by the National Institutes of Health. The government has certain rights in the invention.

### BACKGROUND

Human embryonic stem cells (hESCs) have potential as sources of cells for the treatment for disease and injury (e.g. tissue engineering and reconstruction, diabetes, Parkinson's Disease, leukemia, congestive heart failure, etc.). Features that are important for successful integration of hESC into such therapies include: expansion of hESCs without differentiation (i.e., self-renewal), differentiation of hESCs into a specific cell type or collection of cell types, and functional integration of hESCs or their progeny into existing tissue. Originally, hESCs were grown in monolayer culture with a feeder layer of mouse cells or with conditioned media derived from these feeder cells. Advances in cell culture techniques have led to the development of chemically-defined media and feeder-free hES cell culture systems that employ animal or human-derived extracellular matrix (ECM) proteins to coat the culture substrata. For chemically-defined media, Matrigel™ is a commonly-used ECM analogue. Matrigel™ is an extraction from Engelbreth-Holm-Swarm (EHS) mouse sarcomas that contains not only basement membrane components (laminin, collagen IV, heparin sulfate proteoglycans and entactin), but also matrix degrading enzymes, their inhibitors, numerous growth factors, and a broad variety of other proteins, as recent proteomic data indicate. Matrigel™ represents a poorly defined substrate for precise hESC expansion. Therefore, more recent advances have focused on replacing Matrigel™ and isolated ECM proteins with recombinant proteins, synthetic peptides, and/or polymers.

There is a need in the art for improved culture systems and methods for culturing stem cells, e.g., hESCs, and/or progeny thereof for clinical use.

### LITERATURE

U.S. Pat. No. 7,157,275; U.S. Patent Publication No. 2007/0026518; U.S. Pat. No. 5,863,650; U.S. Patent Publication No. 2004/0001892; and U.S. Patent Publication No. 2007/0099247; WO 2008/118392; Bekos et al. (1995) Langmuir 11:984; Ranieri et al. (1993) J. Biomed. Materials Res. 27:917; Irwin et al. (2011) Biomaterials 32:6912.

### SUMMARY OF THE INVENTION

The present disclosure provides synthetic substrates for long-term culture of stem cells; and methods of use of the synthetic substrates.

### BRIEF DESCRIPTION OF THE DRAWINGS

FIGS. 1A-O depict pluripotency and proliferation of H1s and H9-hOct4-pGZs maintained for 10 passages on N-(3-aminopropyl)methacrylamide (APMAAm).

FIGS. 2A-H depict differentiation of H9-hOct4-pGZ cells after 12 passages on APMAAm or on Matrigel™ substrate.

FIGS. 3A-D depict the kinetics of protein adsorption onto APMAAm from mTeSR™1 medium.

FIG. 4 depicts H9-hOct4-pGZ attachment to APMAAm surfaces after 24 hours in complete mTeSR™1 culture medium or incomplete mTeSR™1 culture medium supplemented with bovine serum albumin (BSA), transforming growth factor-β (TGF-β) or basic fibroblast growth factor (bFGF).

FIGS. 5A and 5B depict analysis of BSA coating; and the effect of BSA on footprint size.

### DEFINITIONS

As used herein, the term “stem cell” refers to an undifferentiated cell that can be induced to proliferate. The stem cell is capable of self-maintenance or self-renewal, meaning that with each cell division, one daughter cell will also be a stem cell. Stem cells can be obtained from embryonic, post-natal, juvenile, or adult tissue. Stem cells can be pluripotent or multipotent. The term “progenitor cell,” as used herein, refers to an undifferentiated cell derived from a stem cell, and is not itself a stem cell. Some progenitor cells can produce progeny that are capable of differentiating into more than one cell type.

Stem cells include pluripotent stem cells, which can form cells of any of the body's tissue lineages: mesoderm, endoderm and ectoderm. Therefore, for example, stem cells can be selected from a human embryonic stem (ES) cell; a human inner cell mass (ICM)/epiblast cell; a human primitive ectoderm cell, a human primitive endoderm cell; a human primitive mesoderm cell; and a human primordial germ (EG) cell. Stem cells also include multipotent stem cells, which can form multiple cell lineages that constitute an entire tissue or tissues, such as but not limited to hematopoietic stem cells or neural precursor cells. Stem cells also include totipotent stem cells, which can form an entire organism. In some embodiments, the stem cell is a partially differentiated or differentiating cell. In some embodiments, the stem cell is an induced pluripotent stem cell (iPSC), which has been reprogrammed or de-differentiated. Stem cells can be obtained from embryonic, fetal or adult tissues.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a synthetic substrate” includes a plurality of such substrates and reference to “the stem cells” includes reference to one or more stem cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable sub-combination. All combinations of the embodiments pertaining to the present disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides synthetic substrates for long-term culture of stem cells;

and methods of use of the synthetic substrates to culture stem cells in vitro. The inventors have found that linking a hydrogel such as aminopropylmethacrylamide (APMAAm) onto a solid support such as polystyrene provides a synthetic substrate that, when used to culture stem cells in a defined culture medium, provides for long-term stem cell culture.

### Synthetic Substrate

A synthetic substrate of the present disclosure comprises a synthetic, non-polypeptide polymer that is covalently linked to a solid support surface.

For example, the solid support surface can comprise a material such as: polyolefins, polystyrenes, “tissue culture treated” polystyrenes, poly(alkyl)methacrylates and poly(alkyl)acrylates, poly(acrylamide), poly(ethylene glycol), poly(N-isopropyl acrylamide), polyacrylonitriles, poly(vinylacetates), poly(vinyl alcohols), chlorine-containing polymers such as poly(vinyl)chloride, polyoxymethylenes, polycarbonates, polyamides, polyimides, polyurethanes, polyvinylidene difluoride (PVDF), phenolics, amino-epoxy resins, polyesters, polyethers, polyethylene terephthalates (PET), polyglycolic acids (PGA) and other degradable polyesters, poly-(p-phenyleneterephthalamides), polyphosphazenes, polypropylenes, and silicone elastomers, as well as copolymers and combinations thereof. In some embodiments, the solid support comprises polystyrene. In some embodiments, the solid support comprises “tissue culture treated” polystyrene, e.g., polystyrene that has been treated with an oxygen plasma to generate oxygen species in the polystyrene. See, e.g., Ramsey et al. (1984) In Vitro 20:802; Beaulieu et al. (2009) Langmuir 25:7169; and Kohen et al. (2009) Biointerphases 4:69.

The synthetic substrate is formed by attaching to a solid support a polymerizable monomer that is adapted to be contacted with the surface of the solid support, in the presence of a grafting reagent or a crosslinking reagent, and to be polymerized upon activation of a photo initiator. Suitable polymerizable monomers include N-(3-aminopropyl)methacrylamide (APMAAm). Other suitable monomers include positively charged monomers such as N-(2-aminoethyl)methacrylate, N-(2-aminoethyl)methacrylamide hydrochloride, N-(2-aminoethyl)methacrylate hydrochloride, monomers containing a primary amine(s), monomers containing terminal primary amines, monomers containing a primary amine linked to a hydrocarbon, and similar monomers.

A suitable monomer (e.g., polymerized APMAAm) can be simultaneously polymerized and covalently linked to the solid support using any of a variety of known chemistries. For example, a photoinitiator, a cross-linking (or grafting) reagent, and a monomer are activated simultaneously to polymerize the monomers and attach the polymer thus formed to the surface of the solid support. For example, a suitable cross-linking (or grafting) reagent is N,N′-methylenebis(acrylamide). Other suitable cross-linking or grafting reagents include other di- or tri-acrylates, e.g., tetra(ethylene glycol) dimethacrylate. Suitable cross-linking reagents include, e.g., 1,1,1-trimethylolpropane triacrylate; dipentaerythritol pentaacrylate; 1,1,1-trimethylolpropane trimethacrylate; pentaerythritol tetraacrylate; pentaerythritol triacrylate; propoxylated (6) trimethylolpropane triacrylate; highly propoxylated (5,5) glyceryl triacrylate; trimethylolpropane trimethacrylate; trimethylolpropane triacrylate; low viscosity trimethylolpropane triacrylate; tris (2-hydroxy ethyl) isocyanurate triacrylate; pentaerythritol triacrylate; ethoxylated (3) trimethylolpropane triacrylate; propoxylated (3) trimethylolpropane triacrylate; ethoxylated (6) trimethylolpropane triacrylate; ethoxylated (9) trimethylolpropane triacrylate; propoxylated (3) glyceryl triacrylate; melamine acrylate; and the like.

A subject synthetic substrate does not include, e.g., peptides, proteins, or Matrigel™. For example, a subject synthetic substrate does not include RGD peptides; proteins comprising RGD; extracellular matrix proteins; and the like.

A subject synthetic substrate can be provided in any of a variety of forms, e.g., a tissue culture dish (e.g., a 5-cm culture dish, a 10-cm culture dish); a multi-well cell culture plate (e.g., a 6-well cell culture plate; etc.); and the like.

### Stem Cell Culture System

The present disclosure provides a stem cell culture system comprising: a) a synthetic stem cell culture substrate, as described above; and b) a serum-free, chemically defined culture medium, wherein the stem cell culture system does not comprise feeder cells.

Stem cells are allowed to adhere to a subject synthetic surface in the presence of a serum-free, defined culture medium, in the absence of feeder cells. Suitable serum-free defined media are known in the art. See, e.g., Akopian et al. (2010) In Vitro Cell. Dev. Biol.—Animal 46:247; Ludwig et al. (2006) Nat. Methods 3:637; and Wang et al. (2007) Blood 110:4111. Suitable serum-free defined media include, e.g., hESF9; mTeSRT™1; STEMPRO; and the like. The composition of such media is known in the art. See, e.g., Akopian et al. (2010) In Vitro Cell. Dev. Biol.—Animal 46:247; Ludwig et al. (2006) Nat. Methods 3:637; and Wang et al. (2007) Blood 110:4111.

A suitable cell culture medium is used for in vitro culture, where a suitable cell culture medium can include one or more of a growth factor, vitamins, serum albumin (e.g., human serum albumin; bovine serum albumin), and the like.

### Stem Cells

Cells that can be cultured on a subject synthetic surface include stem cells, e.g., hematopoietic stem cells, embryonic stem cells, mesenchymal stem cells, neural stem cells, epidermal stem cells, endothelial stem cells, gastrointestinal stem cells, liver stem cells, cord blood stem cells, amniotic fluid stem cells, skeletal muscle stem cells, smooth muscle stem cells (e.g., cardiac smooth muscle stem cells), pancreatic stem cells, olfactory stem cells, hematopoietic stem cells, induced pluripotent stem cells; and the like; as well as differentiated cells that can be cultured in vitro and used in a therapeutic regimen, where such cells include, but are not limited to, keratinocytes, adipocytes, cardiomyocytes, neurons, osteoblasts, pancreatic islet cells, retinal cells, and the like. The cell that is used will depend in part on the nature of the disorder or condition to be treated.

Suitable human embryonic stem (ES) cells include, but are not limited to, any of a variety of available human ES lines, e.g., BG01 (hESBGN-01), BG02 (hESBGN-02), BG03 (hESBGN-03) (BresaGen, Inc.; Athens, Ga.); SA01 (Sahlgrenska 1), SA02 (Sahlgrenska 2) (Cellartis AB; Goeteborg, Sweden); ES01 (HES-1), ES01 (HES-2), ES03 (HES-3), ES04 (HES-4), ES05 (HES-5), ES06 (HES-6) (ES Cell International; Singapore); UC01 (HSF-1), UC06 (HSF-6) (University of California, San Francisco; San Francisco, Calif.); WA01 (H1), WA07 (H7), WA09 (H9), WA09/Oct4D10 (H9-hOct4-pGZ), WA13 (H13), WA14 (H14) (Wisconsin Alumni Research Foundation; WARF; Madison, Wis.). Cell line designations are given as the National Institutes of Health (NIII) code, followed in parentheses by the provider code. See, e.g., U.S. Pat. No. 6,875,607.

Suitable human ES cell lines can be positive for one, two, three, four, five, six, or all seven of the following markers: stage-specific embryonic antigen-3 (SSEA-3); SSEA-4; TRA 1-60; TRA 1-81; Oct-4; GCTM-2; and alkaline phosphatase.

Hematopoietic stem cells (HSCs) are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

Mesenchymal stem cells (MSC), originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

An induced pluripotent stem (iPS) cells is a pluripotent stem cell induced from a somatic cell, e.g., a differentiated somatic cell. iPS cells are capable of self-renewal and differentiation into cell fate-committed stem cells, including neural stem cells, as well as various types of mature cells.

iPS cells can be generated from somatic cells, including skin fibroblasts, using, e.g., known methods. iPS cells produce and express on their cell surface one or more of the following cell surface antigens: SSEA-3, SSEA-4, TRA-1-60, TRA-1-81, TRA-2-49/6E, and Nanog. In some embodiments, iPS cells produce and express on their cell surface SSEA-3, SSEA-4, TRA-1-60, TRA-1-81, TRA-2-49/6E, and Nanog. iPS cells express one or more of the following genes: Oct-3/4, Sox2, Nanog, GDF3, REX1, FGF4, ESG1, DPPA2, DPPA4, and hTERT. In some embodiments, an iPS cell expresses Oct-3/4, Sox2, Nanog, GDF3, REX1, FGF4, ESG1, DPPA2, DPPA4, and hTERT. Methods of generating iPS are known in the art, and any such method can be used to generate iPS. See, e.g., Takahashi and Yamanaka (2006) Cell 126:663-676; Yamanaka et. al. (2007) Nature 448:313-7; Wernig et. al. (2007) Nature 448:318-24; Maherali (2007) Cell Stem Cell 1:55-70; Nakagawa et al. (2008) Nat. Biotechnol. 26:101; Takahashi et al. (2007) Cell 131:861; Takahashi et al. (2007) Nat. Protoc. 2:3081; and Okita et al. (2007 Nature 448:313.

iPS cells can be generated from somatic cells (e.g., skin fibroblasts) by genetically modifying the somatic cells with one or more expression constructs encoding Oct-3/4 and Sox2. In some embodiments, somatic cells are genetically modified with one or more expression constructs comprising nucleotide sequences encoding Oct-3/4, Sox2, c-myc, and K1f4. In some embodiments, somatic cells are genetically modified with one or more expression constructs comprising nucleotide sequences encoding Oct-4, Sox2, Nanog, and LIN28.

### Stem Cell Culture

The present disclosure provides methods for expanding and maintaining stem cells in an undifferentiated state in vitro. In some embodiments, the methods generally involve culturing the stem cells in a serum-free, defined culture medium on a subject synthetic substrate, thereby expanding and maintaining the stem cells in an undifferentiated state. In other embodiments, the methods involve culturing the stem cells in a serum-free, defined culture medium on a subject synthetic substrate, where the synthetic substrate is pre-coated with albumin, where the culturing provides for expanding and maintaining the stem cells in the undifferentiated state, and where the stem cells are cultured in the absence of feeder cells or conditioned medium.

Stem cells can be expanded and maintained in an undifferentiated state using a subject method, for at least 5 passages, at least 10 passages, at least 15 passages, at least 20 passages, at least 25 passages, at least 30 passages, at least 40 passages, at least 50 passages, at least 100 passages, or more than 100 passages.

In the course of culturing stem cells according to the present disclosure, where the stem cells are pluripotent, the stem cells maintain pluripotency throughout the entire culture period (e.g., for at least 5 passages, at least 10 passages, at least 15 passages, at least 20 passages, at least 25 passages, at least 30 passages, at least 40 passages, at least 50 passages, at least 100 passages, or more than 100 passages). Where the stem cells are multipotent, the stem cells maintain multipotency throughout the entire culture period (e.g., for at least 5 passages, at least 10 passages, at least 15 passages, at least 20 passages, at least 25 passages, at least 30 passages, at least 40 passages, at least 50 passages, at least 100 passages, or more than 100 passages). For example, in some cases, the stem cells maintain expression of stem cell markers such as Oct4 (e.g., Oct-3/4), Sox2, and Nanog throughout the culture period. Whether a stem cell maintains expression of Oct4 (Oct-3/4), Sox2, and Nanog can be determined using well-known methods.

Oct-3/4 polypeptides are known in the art and are described in, e.g., U.S. Patent Publication No. 2009/0191159. Nanog polypeptides are known in the art and are described in, e.g., U.S. Patent Publication No. 2009/0047263. See also the following GenBank Accession Nos.: 1) GenBank Accession Nos. NP_{—}002692, NP_{—}001108427; NP_{—}001093427; NP_{—}001009178; and NP_{—}038661 for Oct-3/4; 2) GenBank Accession Nos. AAP49529 and BAC76999, for Nanog. Sox2 polypeptides are known in the art. See, e.g., Kuroda et al. (2005) Mol. Cell. Biol. 25(6):2475-2485. Sox2 amino acid sequences can be found in, e.g., GenBank Accession Nos: NP_{—}003097, NP_{—}001098933, NP_{—}035573, ACA58281, BAA09168, NP_{—}001032751, and NP_{—}648694.

Stem cells cultured according to a method of the present disclosure can have a doubling time of from about 14 hours to about 24 hours, e.g., from about 14 hours to about 16 hours, from about 16 hours to about 18 hours, from about 18 hours to about 20 hours, or from about 20 hours to about 24 hours.

In certain embodiments, the stem cell culture is an essentially homogenous cell culture with respect to a desired characteristic, such as but not limited to karyotype, cell marker expression pattern, or cellular differentiation potential. For example, in some cases, the essentially homogenous cell culture consists of cells that have a normal karyotype. For example, it is contemplated that in such karyotypically essentially homogenous cell cultures, greater than 75%, 80%, 85%, 90%, 91%, 92%, 93%, 94%, 95%, 96%, 97%, 98%, or 99% of metaphases examined will display a normal karyotype.

A culture method of the present disclosure provides for the production of stem cells suitable for use in research and/or clinical applications, where a subject method provides for production of from about 10⁶ to 5×10⁶ stem cells, from about 5×10⁶ to about 10⁷ stem cells, from about 10⁷ to about 5×10⁷ stem cells, from about 5×10⁷ stem cells to about 10⁸ stem cells, from about 10⁸ to about 5×10⁸ stem cells, or from about 5×10⁸ stem cells to about 10⁹ stem cells, or greater than 10⁹ stem cells.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Synthetic Surfaces for Human Embryonic Stem Cell Culture

### Materials and Methods

### Network Polymerization on TCPS

Costar (Corning; Corning, N.Y.) 12-well tissue culture polystyrene (TCPS) plates were used for all cell culture experiments. 12-well plates were activated by a Plasma Preen II 973 Oxygen Plasma (Plasmatic Systems) set at 1 Torr and 150 W for 1 min. Subsequently, hydrogel network coatings were polymerized directly onto the bottom of each well of a 12-well plate via photoinitiated radical addition polymerization. 200 μL of a solution containing monomer, crosslinker, and photoinitiator was pipetted into each well: 0.15 g/mL N-(3-Aminopropyl)methacrylamide hydrochloride (APMAAm; Polysciences, Warrington, Pa.), 0.0015 g/mL N,N-methylenebis(acrylamide) (BIS, Polysciences), and 0.005 g/mL Irgacure 2959 (Ciba) in 97:3 (v/v) water:isopropanol (IPA; Sigma Aldrich). The samples underwent photoinitiated polymerization for 1 min using a UV light source (UV light irradiation of 0.36 mW/cm² at 365 nm). Excess solution was aspirated from wells and then rinsed 3 times in water to remove unreacted materials. Plates were sterilized by soaking in 70:30 (v/v) ethanol:water mix for 20 min followed by 3 rinses in DPBS. All water used in this study was ultra pure ASTM Type I reagent grade water (18 MΩ·cm, pyrogenfree, endotoxin <0.03 EU/mL).

### Network Polymerization on QCM-D Crystals and Si Wafers

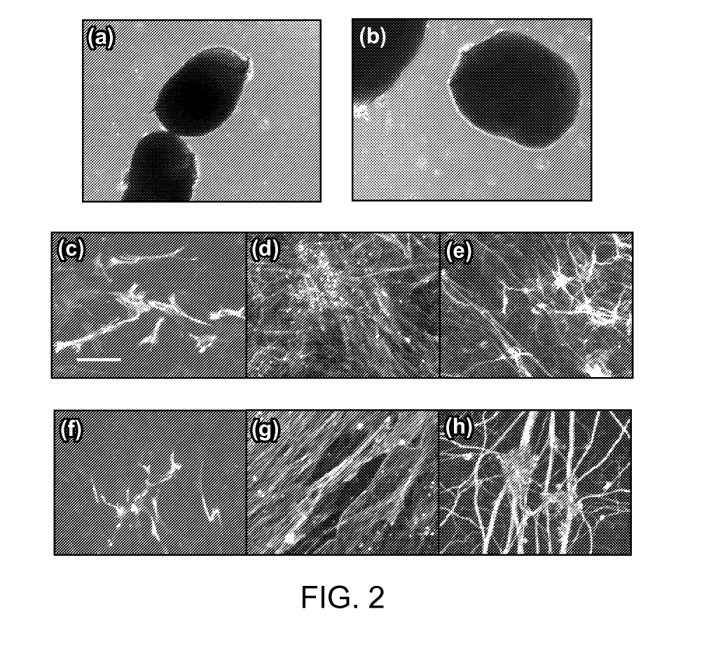

QCM-D sensor crystals (QSX101, Qsense) and Si-wafer pieces (1 cm×1 cm) were cleaned by soaking in water, acetone, and toluene. Polystyrene (PS) films were spin-coated onto QCM-D sensor crystals and Si-wafers at 2000 rpm for 60 secs from a 1% (w/v) PS solution in toluene as described previously.(29) PS films were annealed for 48 hours at 110° C. and subsequently activated by oxygen plasma (Plasmatic Systems) set at 1 Torr and 150 W for 1 min. Hydrogel coatings were photo-polymerized directly onto PS layer by flipping the samples upside down in a 6-well PS plate with 500 μL of monomer solution per well. The solution was identical to above aside from the solvent, which was 100% water. The samples underwent photo-initiated polymerization for 1 min using a UV light source and were rinsed 3× in water. In addition, the presence of the APMAAm surface was validated by XPS measurements on Si-wafer samples (SI FIG. 2) showing an increase in the N peak as shown in the TCPS-APMAAm samples.

### X-ray Photoelectron Spectroscopy (XPS)

APMAAm-modified 12-well as received TCPS plates were sent to NESAC Bio for XPS analysis where all spectra were taken on a Surface Science Instruments S-probe spectrometer with a monochromatized Al Kα X-ray, and a low energy electron flood gun for charge neutralization of non-conducting samples. The samples were floated on double sided tape and run as insulators. Three spots were analyzed on each sample. Samples were analyzed with a pass energy of 150 eV for survey spectra and 50 eV for high resolution scans, and a take-off angle of 55°. Service Physics ESCA2000A Analysis Software was used for peak-fitting. The binding energy scale of the high-resolution spectra was calibrated by setting the primary component to 285.0 eV.

### Contact Angle Goniometry

Water contact angles of as received TCPS and APMAAm substrates (quasi-static advancing (θ_{ADV}^{H}^{₂}^{O}) were measured according to methods previously described(30) using a customized micrometer microscope fitted with a goniometer eyepiece (Gaertner, Chicago, Ill.). All contact angles were measured at ambient temperature to the nearest degree.

### hES Cell Cultures

II1s (31) and H9-hOct4-pGZ (hOct4 promoter driving GFP and Zeo) from Wicell were employed in this work. Human embryonic stem cells (hESCs) were cultured on APMAAm gels and Matrigel™ controls in chemically defined mTeSR™1 medium (Stem Cell Technologies, Vancouver, BC). Cells were fed daily and passaged 1:3-1:6 every 3-5 days by exposure to Collagenase IV (Gibco Invitrogen; Carlsbad, Calif.) at 200 U/mL in Knockout DMEM (KO-DMEM; Gibco Invitrogen) for 5 min at room temperature (RT). Cells were then washed on the dish with Dulbecco's Phosphate Buffered Saline (DPBS; Gibco Invitrogen), followed by mTeSR™1 medium containing 5 μM Rock Inhibitor (Ri; Calbiochem EMD Chemicals). Cells were gently scraped and pipetted into smaller colonies, and passaged 1:4-1:6 in mTeSR™1 medium supplemented with Ri. For controls, matrigel was diluted 1:30 in KO-DMEM at 4° C., allowed to adsorb for more than 10 min at RT, and then aspirated immediately before use.

### hES Differentiation

H1s and H9-hOct4-pGZ were differentiated by normal passaging and suspension in 20% FBS in Knock-out DMEM (Gibco Invitrogen). At Day 8, embryoid bodies (EBs) were plated on gelatin-coated TCPS wells and immunostaining experiments were carried out at day 40.

### Karyotype

After 10 passages on APMAAm, hESCs were passaged back onto matrigel and brought to Children's Hospital Oakland Cytogenics laboratory for karyotyping by GTG-banding.

### Immunostaining and Quantitative Analysis

Samples in 12-well plates were fixed using 4% (v/v) paraformaldehyde in DPBS at 37° C. for 10 min. Samples were then rinsed 3× in phosphate buffered saline (PBS) and kept at 4° C. Cells were permeabilized with 0.1% Triton-X (Sigma) for 10 min; for intracellular markers, cells were further permeabilized with 0.5% sodium dodecyl sulfate (SDS) for 5 min. Cells were incubated with a 1:100 dilution of primary antibody [Mouse Anti-Oct-4 IgG (Santa Cruz Biotechnology); Mouse Anti-SSEA-4 IgG (Millipore); Mouse Anti-Tra-1-60 IgM (Millipore); Rabbit Anti-Desmin IgG (Thermo Scientific); Rabbit Anti-(-Smooth Muscle Actin IgG (Millipore); Mouse Anti-Human B Tubuliin III IgG (Millipore)] overnight at 4° C. The next day, cells were incubated with an appropriate Alexa Fluor secondary antibody 1:300 for 1 hr at RT [Goat Anti-Mouse AlexaFluor 488 IgG (Molecular Probes), Goat Anti-Mouse AlexaFluor 488 IgM (Molecular Probes), Goat Anti-Rabbit AlexaFluor 546 IgG (Molecular Probes)]. Finally, cell nuclei were stained with 4′,6-diamino-2-diamidino-2-phenylindole, dilactate (DAPI; Molecular Probes) for 5 min at RT. All staining steps were followed by 3 washes in PBS. Cells were visualized immediately as follows: Epifluorescent imaging (Axiovert), whole plate imaging (ImageXpressMicroscope), and Confocal imaging (Zeiss). For quantitative image analysis, the Metamorph software was used and the ‘Cell scoring’ algorithm was applied. Briefly, the cell nuclei were identified by DAPI staining and the corresponding area positive for a specific wavelength (i.e. AlexaFluor 488, AlexaFluor 546) was measured. The percent of cells that were associated with a positive stain was quantified for 49 different sites (1000 μm²/site) on each well. Data is presented as the mean percentage of positive cells on all sites±SEM.

### Quantitative RT-PCR

RNA and cDNA were attained from cells using Taqman Fast Cells-to-C_{T} Kit (Applied Biosystems) according to the manufacturer's instructions for n=3 biological replicates. cDNA and reverse transcription-polymerase chain reaction (RT-PCR) reactions were performed with a StepOnePlus instrument (Applied Biosystems) using TaqMan Fast Universal PCR Master Mix, and TaqMan Gene Expression Assays (GAPDH HS9999905_m1; Oct4 Hs00742896_s1; Nanog Hs02387400_g1; Sox2 Hs00602736_s1) according to the manufacturer's instructions. Quartz Crystal Microbalance with Dissipation (QCM-D) experiments

In a QCM-D, an AC voltage is pulsed across an AT-cut piezoelectric quartz crystal, causing it to oscillate in shear mode at its resonant frequency. The resonant frequency of the crystal is recorded in real time and depends on the total oscillating mass and the intrinsic properties of the quartz crystal. The Sauerbrey relationship(32) states that a change in the mass (ΔM) of a film or adlayer is directly proportional to a change in the normalized resonant frequency of the crystal (ΔF):

ΔzM=−C*ΔF/n

where C is the mass sensitivity constant of −17.7 ng cm⁻² Hz⁻¹ and n is the overtone number. The ΔF is due to the change in total coupled mass, including hydrodynamically coupled water and water associated with adsorbed molecules. The dampening of the shear wave is also recorded simultaneously with the resonant frequency of the crystal as the dissipation factor (D), which is the ratio of the dissipated energy to the stored energy. For this work, APMAAm-modified sensor crystals were loaded into the QCM-D (E4, Biolin Scientific, Sweden) and solutions were flowed over the surface of the crystal at 400 μL/min using a Peristaltic pump (Ismatec IPC-N4; Glattbrugg, Switzerland). F and D were recorded in real time at 4 different overtones: n=1, 3, 5, and 7. Initially, DPBS flowed over the APMAAm surface to establish a baseline for the frequency (F) and dissipation (D) values of the crystal. Once F and D were stabilized, different solutions were introduced sequentially until the measurement reached equilibrium. All calculations were done with n=7 overtone since it contained the least amount of noise.

### Cell Attachment Studies

hESCs were passaged normally and plated in the specified media. After 24 h, hESCs were washed in PBS and frozen at −20° C. immediately. Cyquant (Molecular Probes) was used to quantify cell attachment according to the manufacturer's instructions. Briefly, cells were simultaneously lysed and incubated with a proprietary green fluorescent dye, which fluoresces when bound to nucleic acids for 5 min. After the incubation time, solution was pipetted onto a 96-well black plate (Costar) and the fluorescence was measured employing a Spectramax GeminiXS spectrofluorometer (Molecular Devices, CA; ex/em/cutoff, 480/520/515 nm). The population doubling time (PDT) was calculated according to the following equation: (33)

PDT=(T₂−T₁)/┌3.32*(log N₂−log N₁)┐,

Where T₁ was day 1, T₂ was day 4, N₁ was the number of cells at T₁, and N₂ was the number of cells at T₂.

### BSA Spreading Method

PS films were spin-coated onto Au QCMD sensor crystals, and annealed for 48 hours. APMAAm surfaces were modified onto PS film, stored in water for two days, then dried. Measurements were taken with E4 of 4 BSA solutions simultaneously: 0.01 mg/mL, 0.025 mg/mL, 0.05 mg/mL, and 0.1 mg/mL. PBS, pH 7.4, was introduced and allowed to stabilize for 10 minutes. All four protein solutions were introduced simultaneously into four separate chambers at 200 μL/minute for about 3.5 hours. PBS, pH 7.4, was introduces and allowed to stabilize for 10 minutes. For calculations of footprint size, F7/7 was used with the Sauerbrey relationship.

### Statistics

All data were expressed as the average of at least three replicate experiments ± the standard error of the mean. Statistical comparisons were performed by ANOVA (P<0.05) followed by Holms t-tests (P<0.05) for significance.

### Results

We have developed a synthetic polymer interface for the long-term self-renewal of hESCs. The hydrogel network coating is comprised of aminopropylmethacrylamide (APMAAm) monomer and N,N-methylenebis(acrylamide)(bis) crosslinker that was grafted to standard tissue culture polystyrene (TCPS) dishes via photoinitiated addition polymerization.

The structures of N-(3-aminopropyl)methacrylamide hydrochloride (left) and N,N′-methylenebisacrylamide (right) are shown below.

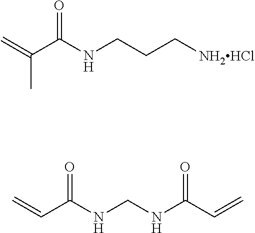

We verified the polymerization reaction with X-ray photoelectron spectroscopy (XPS) and contact angle goniometry. The photoemission data showed consistently increased N and corresponding decreased C on the APMAAm samples compared to the as received TCPS control. After polymerization, water advancing water contact angles (θ_{ADV}^{H}^{₂}^{O}) changed from 72.6±0.3° to 35.3±0.3°, which is in agreement with previously published data for a self-assembled monolayer (SAM) alkanethiolates and organosilanes presenting a terminal amine.(23, 24). Table 1 shows chemical composition of the APMAAm surface by XPS shows the introduction of a N peak with modification. Table 2 shows results of high resolution C1s XPS analysis on APMAAm and PS controls.

TABLE 1
XPS Analysis (Percent Composition)

| | C 1s | O 1s | N 1s | Cl 2p | Si 2p |
|---|---|---|---|---|---|
| APMAAm | 82.9 ± 1.0 | 13.8 ± 0.4 | 2.9 ± 0.4 | 0.3 ± 0.1 | 0.2 ± 0.3 |
| PS control | 84.7 ± 0.4 | 15.3 ± 0.4 | 0.0 ± 0.0 | 0.0 ± 0.0 | 0.0 ± 0.0 |

TABLE 2
XPS High Resolution C 1s Results

| | | | | | Shake- |
|---|---|---|---|---|---|
| | C—C | C—O,N | C═O | O—C═O | up |
| APMAAm | 82.1 ± 3.2 | 11.4 ± 2.7 | 4.6 ± 0.7 | 1.9 ± 1.7 | 0.0 ± 0.0 |
| PS control | 76.2 ± 7.7 | 10.6 ± 1.6 | 8.6 ± 4.2 | 2.7 ± 2.1 | 1.8 ± 1.6 |

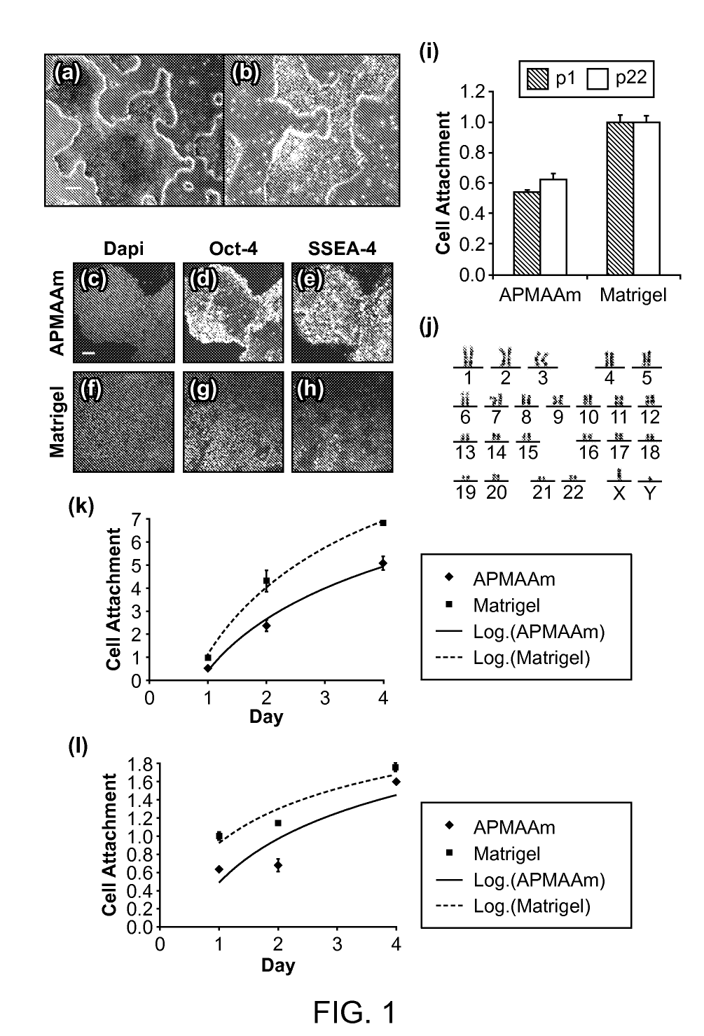

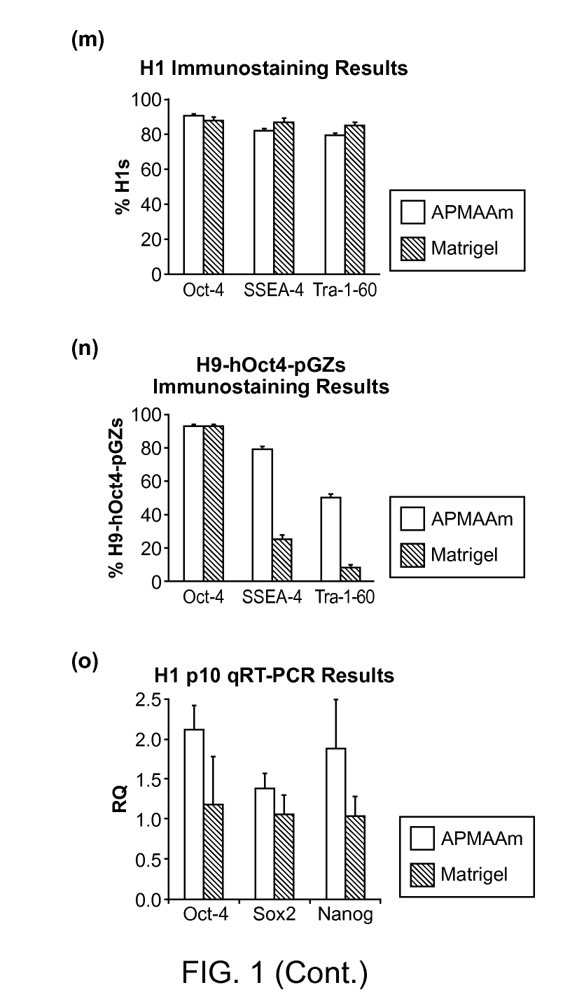

The APMAAm networks maintained hESC pluripotency for long-term culture. We cultured both H1s and H9-hOct4-pGZ cell lines on APMAAm substrates for 10 passages (p10) in chemically-defined mTeSR™1 media, and characterized the pluripotency of both cell lines compared to Matrigel™-coated substrata. Throughout 10 passages, H1s and H9-hOct4-pGZs maintained typical stem cell morphology and grew in colonies similar to Matrigel™ controls (FIG. 1a,b). The pluripotency of both lines was confirmed via immunostaining, where the expression levels of pluripotency markers of II1s and H9-hOct4-pGZs were similar to Matrigel™ controls (FIG. 1c-h). Quantitative analysis of the staining indicated the APMAAm networks were robust in maintaining pluripotent markers for H1s (FIG. 1m). For the H9-hOct4-pGZs, APMAAm interfaces maintained pluripotent markers to a greater degree than Matrigel™ (FIG. 1n); due to the gene knock-in, these cells spontaneously differentiate and are difficult to maintain at high passages in culture. In addition, H1s showed expression of pluripotency genes on APMAAm networks similar to Matrigel™ (FIG. 1o). Finally, the karyotype of both cell lines was normal after culture on APMAAm networks (FIG. 1j shows H1 karyotype).

Interfaces of APMAAm networks were as effective as Matrigel™-coated substrata in supporting the proliferation of hESCs. H1s and H9-hOct4-pGZs were cultured on APMAAm substrates in mTeSR™1 media and compared to Matrigel™. On the first passage from Matrigel™, H9-hOct4-pGZ attachment on APMAAm was approximately half of that on Matrigel™ (FIG. 1i). However, the hESCs adapted to the APMAAm substrate, where the number of cells attached to APMAAm increased to 63.3±0.04% relative to adhesion on Matrigel™ at p22 (FIG. 1i). Although there were initially less cells attached on APMAAm, the proliferation of the H9-hOct4-pGZs was similar to Matrigel™ at both passages 1 and 22 (FIG. 1k & 1). The population doubling time of the H9-hOct4-pGZs at passage 1 on APMAAm was 22.4 h, compared with 26.1 h on Matrigel™. At passage 22 on the APMAAm (total passage number of 80), the H9-hOct4-pGZs slowed their proliferation to a population doubling time of 54.0 h, compared with 88.8 h on Matrigel™.

FIGS. 1A-O. Pluripotency of H1s and H9-hOct4-pGZs was maintained for 10 passages on APMAAm. Representative phase contrast images of H1s on (a) APMAAm and (b) Matrigel™-coated substrates at p8 (scale bar=25 μm). Representative confocal image stacks at p10 of H1 colonies on APMAAm stained with (c) Dapi, (d) Oct-4, and (e) SSEA-4; compared with H1 colonies on Matrigel™ stained with (f) Dapi, (g) Oct-4, (h) SSEA-4 (scale bar=10 μm). (i) H9-hOct4-pGZ cell attachment at p1 and p22 onto APMAAm and Matrigel™ normalized to Matrigel™. (j) H1s and H9-hOct4-pGZs had a normal karyotype after p10 on APMAAm surfaces (H1 karyotype shown). (k) H9-hOct4-pGZ proliferation at pl on APMAAm and Matrigel™ surfaces (normalized to Matrigel™ d1). (1) H9-hOct4-pGZ proliferation at p22 on APMAAm and Matrigel™ (normalized to Matrigel d1). Quantitative immunostaining of (m) H1s and (n) H9-hOct4-pGZ for Oct4, SSEA-4, and TRA-1-60 after p10 on APMAAm and Matrigel™. (o) Quantitative RT-PCR results for pluripotent markers Oct4, Sox2, and Nanog at p10 (H1).

Human ES cells cultured on APMAAm interfaces were differentiated into embryoid bodies (EBs) to demonstrate formation into all three germ layers. H9-hOct4-pGZs cultured on APMAAm for 12 passages formed nearly spherical EBs with typical morphology (FIG. 2a). At Day 40 after EB formation, immunostaining results indicated the formation of all three germ layers in EBs on Matrigel™ and APMAAm samples (FIG. 2b) indicating that the cells retain the multilineage potential after culture on APMAAm surfaces. We believe this same system has the potential to be used for both self-renewal of hiPS, and directed differentiation of hESCs and hiPS into specific lineages under the appropriate media conditions.

FIGS. 2A-H. After 12 passages on APMAAm and Matrigel, H9-hOct4-pGZ cells were differentiated into EBs. Phase contrast images of Day 11 EBs from (a) APMAAm and (b) Matrigel surfaces after p10. Immunostaining for germ layer markers on day 40 for EBs derived from cells cultured on APMAAm surfaces: (c) Desmin (endoderm); (d) Smooth Muscle Actin (SMA; mesoderm); (e) βTubulin III (ectoderm); and, on Matrigel™: (f) Desmin; (g) SMA (mesoderm); and (h) β Tubulin III (scale bars=10 μm).

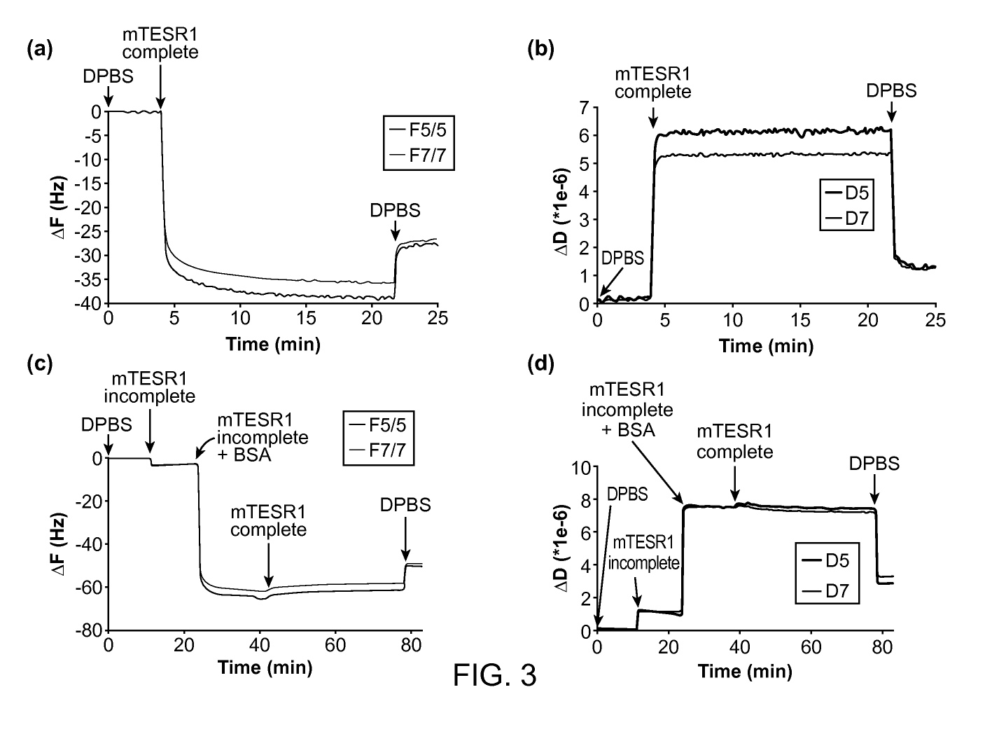

As the interface was not functionalized with peptides or proteins to promote cell adhesion, we sought to understand the mechanism for hESC attachment to the APMAAm networks. We employed a Quartz Crystal Microbalance with Dissipation (QCM-D; Biolin Scientific; described in detail in methods) to record molecular adsorption from the mTeSR™1 media to the APMAAm interface in real time. When the mTeSR™1 media was introduced to the QCM-D chamber, there was a decrease in the frequency of the crystal (ΔF; FIG. 3a) and an increase in the dissipation factor (ΔD; FIG. 3b). As the ΔD was low and the ΔF curves of the different overtones were nearly overlapping, we treated the adsorbed film as a rigid elastic film. According to the Sauerbrey relationship, the observed decrease in F is equivalent to the adsorption of a layer of ˜620 ng/cm² within 15 minutes of exposure (FIG. 3a). This mass includes any coupled water, which can contribute significantly to the mass of the adsorbed layer.(25) Next, approximately 26% of the layer was desorbed when DPBS was introduced into the chamber following the mTeSR™1 media, resulting in a final film mass of ˜460 ng/cm². Due to the significant mass of the adsorbed film and the classic Langmuir adsorption isotherm obtained, we hypothesized that this layer was comprised of proteins from the mTeSR™1 media.

FIGS. 3A-D. Protein adsorption to APMAAm from mTESR™1 allows for hESC attachment. Kinetics of protein adsorption from mTESR™1 complete media onto the APMAAm as determined by QCM-D. Initially APMAAm-modified sensor crystals were baselined in DPBS, where no adsorption to the surface occurs. Next, complete mTESR™1 media was introduced into the chamber and resulted in a decrease in (a) frequency (ΔF) of the crystal (corresponding to an increase in adsorbed mass) and an increase in the (b) dissipation factor (D). After rinsing, the final mass of the adsorbed film was 460 ng/cm². QCM-D measurements of the (c) ΔF and (d) ΔD with the adsorption of a layer of BSA onto the APMAAm from incomplete mTeSR™1 media supplemented with only BSA. Subsequent introduction of complete mTeSR™1, followed by PBS, leads to a stable adsorbed protein film of 810 ng/cm².

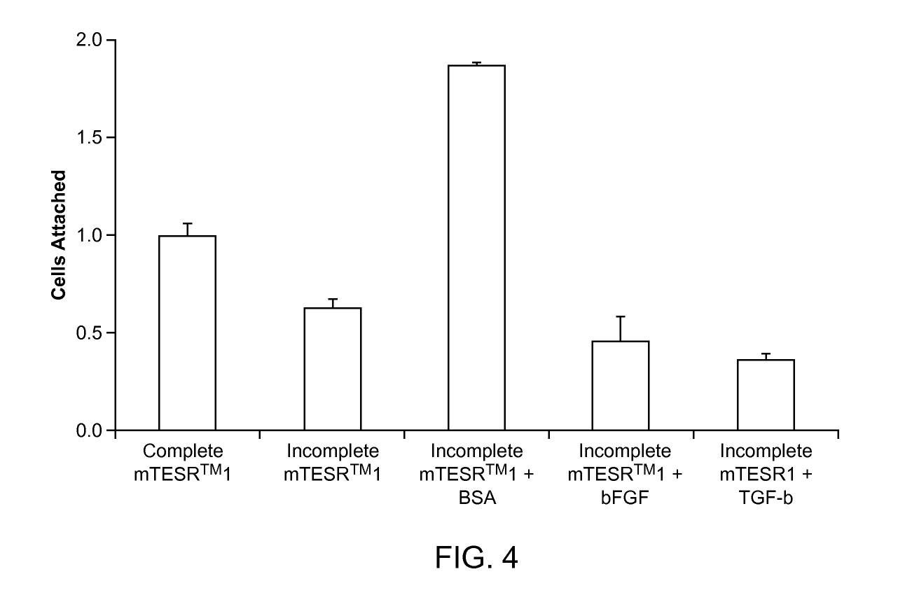

With evidence that a macromolecular layer was adsorbing to the APMAAm interface, we sought to understand which molecule in this layer promoted hESC attachment. The complete mTeSR™1 media contained only 3 proteins; bovine serum albumin (BSA; [12.9 mg/mL]), transforming growth factor beta (TGF-β; ┌1 ng/mL┐), and basic fibroblastic growth factor (bFGF; └0.1 μg/mL┘). We explored the role of these proteins in H9-hOct4-pGZ attachment to APMAAm by performing cell attachment studies with imTeSR™1 (incomplete; basal media without the frozen supplement) supplemented with the aforementioned individual proteins. The number of cells attached after 24 hours in different media on APMAAm is shown in FIG. 4. The addition of BSA to imTeSR™1 resulted in four times the number of hESCs attached compared to the imTeSR™1 with either bFGF or TGF-β. Furthermore, imTeSR™1 with BSA alone led to nearly twice the hESC attachment compared to the complete mTeSR™1 media. These observations indicated that other molecules in the complete media could compete for BSA binding sites on the APMAAm network, limiting hESCs adhesion to the surface.

FIG. 4. H9-hOct4-pGZ attachment to APMAAm surfaces after 24 h in complete mTeSR™1 and incomplete mTeSR™1 supplemented with either BSA, TGF-β or bFGF, (normalized to complete mTeSR™1). The addition of BSA to imTeSR™1 led to more than four times the number of hESCs attached compared to the imTeSR™1 with either bFGF or TGF-β.

In order to further analyze the BSA layer adsorbed to the APMAAm surface, we performed additional protein adsorption experiments with the QCM-D. In these experiments, we employed the incomplete media similarly to the cell attachment experiments. First, imTeSRT™1 was introduced into the chamber, resulting in the adsorption of ˜10 ng/cm² layer (FIG. 3c). This layer was likely comprised of amino acids and lipids present in the basal media. When the imTeSR™1 with BSA was sequentially introduced into the chamber, a layer of ˜1080 ng/cm² of BSA adsorbed onto the APMAAm within 15 min, a greater mass than from the complete media. Assuming a maximum-packed layer of end-on adsorbed BSA (˜14 nm×4 nm×4 nm), a monolayer of BSA corresponds to a mass of ˜790 ng/cm²; however this does not include the mass of coupled water. The change in dissipation (AD) was 7.5×10⁻⁶ (FIG. 3d), indicating the protein layer was less rigid than the film adsorbed from the complete media (AD of 5.0×10⁻⁶). After the preadsorption of the BSA, very little desorption occurred when complete mTeSR™1 media was sequentially introduced into the chamber. However, when DPBS was introduced into the chamber, 25% of the film was desorbed. The final surface density of the film was ˜810 ng/cm², compared to ˜460 ng/cm² of protein from the complete media. Collectively, these data suggest that the layer adsorbing to the APMAAm is primarily BSA, although the lower mass adsorbed from the complete media indicated competition from smaller molecules occupying adsorption sites on the surface. This conclusion is further supported by the cell attachment data, where the imTeSR™1 with BSA led to a higher level of cell attachment than the complete media where less BSA binding sites were available.

Protein adsorption and cell attachment studies identified BSA as a key component in the mTESR™1 media allowing for hESC attachment to APMAAm interfaces. In contrast, BSA is a large serum protein (MW=66 kDa) traditionally used in immunoassays as a blocking protein(26) due to its stability and lack of involvement in most biochemical reactions.

However, Bekos, et al.,(27) and Ranieri, et al.,(28) observed cell attachment mediated by BSA adsorption on aminated polymer films. It was reported that BSA adsorbed to a poly(tetrafluoroethylene-co-hexafluoropropylene) that has been treated with radiofrequency glow charge plasma (FEP-OH), and subsequently modified with an (aminopropyl)trimethoxysilane (resulting in an aminated surface) showed significantly increased mouse neuroblastoma cell (NB2a) and rat endothelial cell (REC) attachment compared with the hydroxylated FEP-OH surface. In addition, by attaching fluorescent markers that detected protein unfolding, it was shown that unlike adsorption on hydroxyl surfaces, BSA unfolded on the aminated surfaces. It was hypothesized that with unfolding, BSA presented an internal hydrophobic domain that either through charge-charge interactions or some unknown binding sequence allowed for N2k and REC attachment.

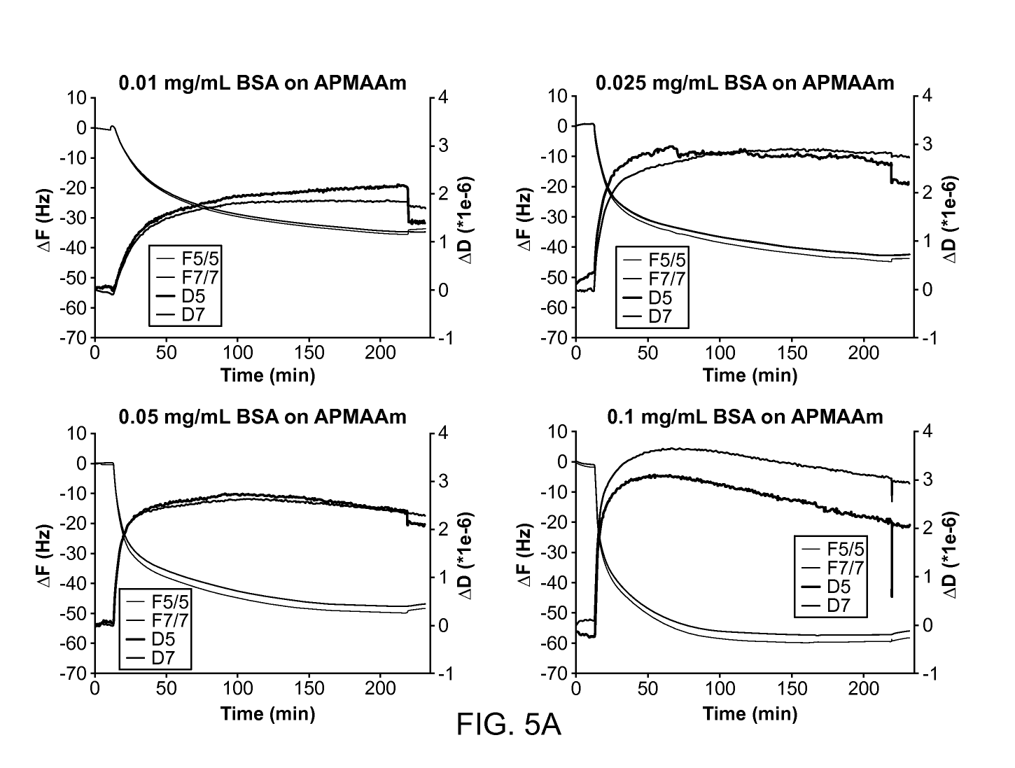

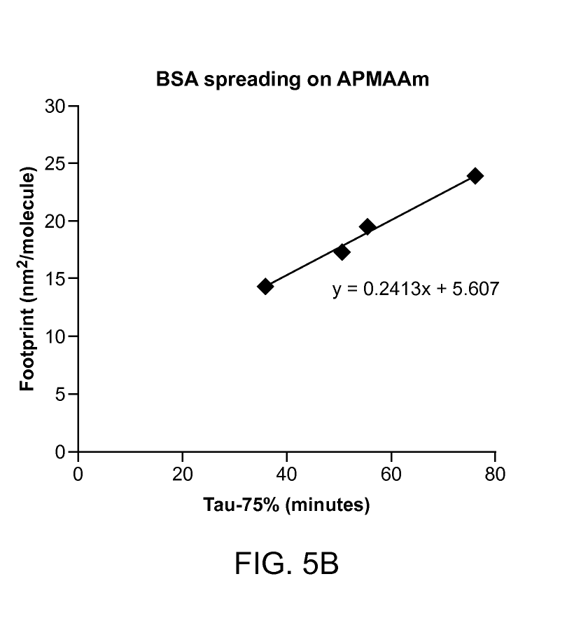

To further understand the behavior of BSA on APMAAm surfaces, BSA spreading experiments were conducted, as described elsewhere (Wertz, M. Santore, Effect of surface hydrophobicity on adsorption and relaxation kinetics of albumin and fibrinogen: Single-species and competitive behavior. Langmuir 17:3006 (2001)) on APMAAm surfaces and analyzed employing a QCM-D. The data are shown in FIGS. 5A and 5B. BSA solutions of different concentrations in PBS were flowed over the APMAAm surface at 200 μL/min. The footprint size of BSA molecules was determined at τ-75%, defined as the time at which 75% of surface saturation was reached. Wertz (2001), supra. The different concentrations of BSA in PBS resulted in different τ-75% values (FIG. 5B), which were used to calculate the different footprint sizes (nm²/molecule). At lower concentrations, the larger footprint sizes indicate that BSA spread on the APMAAm surfaces. However, when BSA was adsorbed from incomplete media (i.e., imTeSR1), it showed the highest mass of albumin adsorbed (FIG. 3a), greatest number of cells attached (FIG. 4), and the smallest footprint size. The dimensions of the footprint size indicate end-on adsorption of the molecule and no change in its conformation upon adsorption. Thus, BSA adsorption enhances hESC adhesion, and the level of cell adhesion in improved when the molecule does not change conformation upon adsorption. This is in contrast to observations from Bekos, et al.,(27) and Ranieri, et al.,(28). The data indicate that BSA adsorbed to APMAAm surfaces promotes the undifferentiated growth of hESCs and maintains their pluripotency.

We have developed a synthetic and defined culture system that allows for long-term hESC growth and self-renewal. The primary advantage of this system is it does not require attachment of peptides or proteins to promote cell attachment, is scalable, low cost, and is free of complex, undefined culture conditions.

### REFERENCES

1. T. E. Ludwig et al., Nature Methods 3, 637 (2006).
2. B. S. Mallon, K. Y. Park, K. G. Chen, R. S. Hamilton, R. D. G. McKay, International Journal of Biochemistry & Cell Biology 38, 1063 (2006).
3. L. Wang et al., Blood 110, 4111 (2007).
4. C. H. Xu et al., Nature Biotechnology 19, 971 (2001).
5. Y. J. Li, E. H. Chung, R. T. Rodriguez, M. T. Firpo, K. E. Healy, Journal of Biomedical Materials Research Part A 79A, 1 (2006).
6. G. M. Beattie et al., Stem Cells 23, 489 (2005).
7. L. Xiao, X. Yuan, S. J. Sharkis, Stem Cells 24, 1476 (2006).
8. S. Yao et al., Proceedings of the National Academy of Sciences of the United States of America 103, 6907 (2006).
9. J. M. Fletcher et al., Cloning and Stem Cells 8, 319 (2006).
10. Y. X. Liu et al., Biochemical and Biophysical Research Communications 346, 131 (2006).
11. H. K. Kleinman et al., Biochemistry 21, 6188 (1982).
12. K. C. Hansen et al., Mol Cell Proteomics 8, 1648 (July 2009).
13. T. E. Ludwig et al., Nature Biotechnology 24, 185 (2006).
14. Y. Meng et al., Faseb Journal 24, 1056.
15. R. Derda et al., Acs Chemical Biology 2, 347 (2007).
16. R. Derda et al., Journal of the American Chemical Society 132, 1289 (February 2010).
17. Y. Mei et al., Nature Materials 9, 768.
18. Z. Melkoumian et al., Nature Biotechnology 28, 606 (2010).
19. R. K. Klim, L. Li, P. J. Wrighton, M. S. Piekarczyk, L. L. Kiessling, Nature Methods 7, 989 (2010).
20. P. Kolhar, V. R. Kotamraju, S. T. Hikita, D. O. Clegg, E. Ruoslahti, Journal of Biotechnology 146, 143 (2010).
21. L. G. Villa-Diaz et al., Nature Biotechnology 28, 581 (2010).
22. D. A. Brafman et al., Biomaterials 31, 9135 (2010).
23. K. E. Healy et al., Biomaterials 17, 195 (January 1996).
24. D. A. Stenger et al., Journal of the American Chemical Society 114, 8435 (1992).
25. F. Hook et al., Analytical Chemistry 73, 5796 (2001).
26. R. A. Smith, M. W. Mosesson, A. U. Daniels, T. K. Gartner, Journal of Materials Science-Materials in Medicine 11, 279 (2000).
27. E. J. Bekos, J. P. Ranieri, P. Aebischer, J. A. Gardella, F. V. Bright, Langmuir 11, 984 (1995).
28. J. P. Ranieri et al., Journal of Biomedical Materials Research 27, 917 (1993).
29. N. T. Kohen, L. E. Little, K. E. Healy, Biointerphases 4, 69 (2009).
30. J. P. Bearinger et al., Langmuir 13, 5175 (1997).
31. J. A. Thomson et al., Science 282, 1145 (1998).
32. G. Sauerbrey, Z Phys 155 (1959).
33. J. Davis, Basic Cell Culture: a Practical Approach (Oxford, New York, 1994), pp.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A synthetic stem cell culture substrate comprising a synthetic, non-polypeptide polymer covalently linked to a solid support surface, wherein the synthetic cell culture substrate does not comprise a peptide or a polypeptide.

2. The synthetic cell culture of claim 1, wherein the synthetic, non-polypeptide polymer is a polymer of aminopropylmethacrylamide (APMAAm) or 2-aminoethyl methacrylate.

3. The synthetic cell culture of claim 1, wherein the solid support surface has been treated to generate oxygen species.

4. The synthetic cell culture of claim 3, wherein the solid support surface is polystyrene.

5. A stem cell culture system comprising:
a) the synthetic stem cell culture substrate of claim 1; and
b) a serum-free, chemically defined culture medium, wherein the stem cell culture system does not comprise feeder cells.

6. The stem cell culture system of claim 5, wherein the serum-free, chemically defined culture medium comprises serum albumin.

7. A method of expanding and maintaining stem cells in an undifferentiated state, the method comprising culturing the stem cells in a serum-free, defined culture medium on the synthetic substrate of claim 1, thereby expanding and maintaining the stem cells in the undifferentiated state, wherein the stem cells are cultured in the absence of feeder cells or conditioned medium.

8. The method of claim 7, wherein the stem cells are embryonic stem cells, multipotent stem cells, or induced pluripotent stem cells.

9. The method of claim 7, wherein the stem cells are derived from a human or a non-human primate.

10. The method of claim 7, wherein the stem cells are embryonic stem cells.

11. The method of claim 7, wherein the stem cells are adult stem cells.

12. The method of claim 7, wherein said culturing is repeated through at least 5 passages, at least 10 passages, or at least 20 passages.

13. A method of expanding and maintaining stem cells in an undifferentiated state, the method comprising culturing the stem cells in a serum-free, defined culture medium on the synthetic substrate of claim 1, wherein the synthetic substrate is pre-coated with albumin, wherein said culturing provides for expanding and maintaining the stem cells in the undifferentiated state, and wherein the stem cells are cultured in the absence of feeder cells or conditioned medium.

14. The method of claim 13, wherein the albumin is bovine serum albumin.

15. The method of claim 13, wherein the stem cells are embryonic stem cells, multipotent stem cells, or induced pluripotent stem cells.

16. The method of claim 13, wherein the stem cells are derived from a human or a non-human primate.

17. The method of claim 13, wherein the stem cells are embryonic stem cells.

18. The method of claim 13, wherein the stem cells are adult stem cells.

19. The method of claim 13, wherein said culturing is repeated through at least 5 passages, at least 10 passages, or at least 20 passages.
